(19) United States
(12) Plant Patent Application Publication
(10) Pub. No.: US 2017/0325374 P1
(43) Pub. Date: Nov. 9, 2017

(54) PERICALLIS PLANT NAMED 'DPRIMBLUBR'

(50) ;

(71) Applicant: Gavriel Danziger, Beit Dagan (IL)

(72) Inventor: Gavriel Danziger, Beit Dagan (IL)

(21) Appl. No.: 14/999,429

(22) Filed: May 3, 2016

(51) Int. Cl. A01H 5/02

(52) U.S. Cl. CPC A01H 5/025
USPC PLT/263.1

(57) ABSTRACT

A new and distinct Pericallis cultivar named ‘DPRIMBLUBR’ is disclosed, characterized by a very compact and mounded habit with dense branching, early flowering and tolerance for powdery mildew. Additionally all flowers are at the same height. The new variety is a Pericallis, normally produced as an outdoor garden or container plant.

Latin name of the genus and species: Pericallis hybrida.

Variety denomination: ‘DPRIMBLUBR’.

### BACKGROUND OF THE INVENTION

The new Pericallis cultivar is a product of a planned breeding program conducted by the inventor, Gavriel Danziger in Moshav Mishmar Hashiva, Israel. The objective of the breeding program was to produce new Pericallis varieties. The open pollination resulting in this new variety was made during May of 2012.

The seed parent is the unpatented, propriety variety referred to as Pericallis ‘CN-12-251’. The pollen parent is unknown as it was an open pollination breeding program. The new variety was discovered in December of 2013 by the inventor in a group of seedlings resulting from the 2012 crossing, in a greenhouse in Moshav Mishmar Hashiva, Israel.

Asexual reproduction of the new cultivar ‘DPRIMBLUBR’ was first performed by terminal vegetative cuttings during January of 2014 at a greenhouse in Moshav Mishmar Hashiva, Israel. Subsequent propagation by vegetative cuttings has shown that the unique features of this cultivar are stable and reproduced true to type in multiple generations.

### SUMMARY OF THE INVENTION

The cultivar ‘DPRIMBLUBR’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DPRIMBLUBR’ These characteristics in combination distinguish ‘DPRIMBLUBR’ as a new and distinct Pericallis cultivar:
1. Very compact and mounded habit.
2. Early flowering.
3. All the flowers occurring evenly at the same height.
4. Tolerance to powdery mildew.

### PARENT COMPARISON

Plants of the new cultivar ‘DPRIMBLUBR’ are similar to plants of the seed parent, most horticultural characteristics, however, plants of the new cultivar ‘DPRIMBLUBR’ differ in the following;
1. More compact habit than the seed parent.
2. Blooms earlier than the seed parent.
3. More floriferous than the seed parent.

### COMMERCIAL COMPARISON

Plants of the new cultivar ‘DPRIMBLUBR’ can be compared to the unpatented commercial variety Pericallis ‘SUSENERABU’, U.S. Plant Pat. No. 16,357. These varieties are similar in most horticultural characteristics; however, ‘DPRIMBLUBR’ differs in the following:
1. Plants of ‘DPRIMBLUBR’ have smaller flowers than plants of ‘SUSENERABU’.
2. Plants of ‘DPRIMBLUBR’ have a violet blue with white center flower color compared to the light blue flower color of ‘SUSENERABU’.
3. Plants of ‘DPRIMBLUBR’ have smaller ray florets than ‘SUSENERABU’.

Plants of the new cultivar ‘DPRIMBLUBR’ can also be compared to the unpatented commercial variety Pericallis ‘Spring Touch Grape’. These varieties are similar in most horticultural characteristics; however, ‘DPRIMBLUBR’ differs in the following:
1. Plants of ‘DPRIMBLUBR’ have smaller flowers than plants of ‘Spring Touch Grape’.
2. Plants of ‘DPRIMBLUBR’ are more compact than plants of ‘Spring Touch Grape’.
3. Plants of ‘DPRIMBLUBR’ bloom earlier than plants of ‘Spring Touch Grape’.
4. Plants of ‘DPRIMBLUBR’ have a violet blue with white centered flower compared to the dark purple flower color of ‘Spring Touch Grape’.

### BRIEF DESCRIPTION OF THE PHOTOGRAPHS

The accompanying photograph in FIG. 1 illustrates in full color a typical plant of ‘DPRIMBLUBR’ grown in 13 cm pots outdoors in Moshav Mishmar Hashiva, Israel.

FIG. 2 illustrates in full color multiple typical inflorescence of ‘DPRIMBLUBR’. Age of the plant photographed is approximately 11 weeks from a rooted cutting in a 13 cm pot. The photographs were taken using conventional techniques and although colors may appear different from actual colors due to light reflectance it is as accurate as possible by conventional photographic techniques.

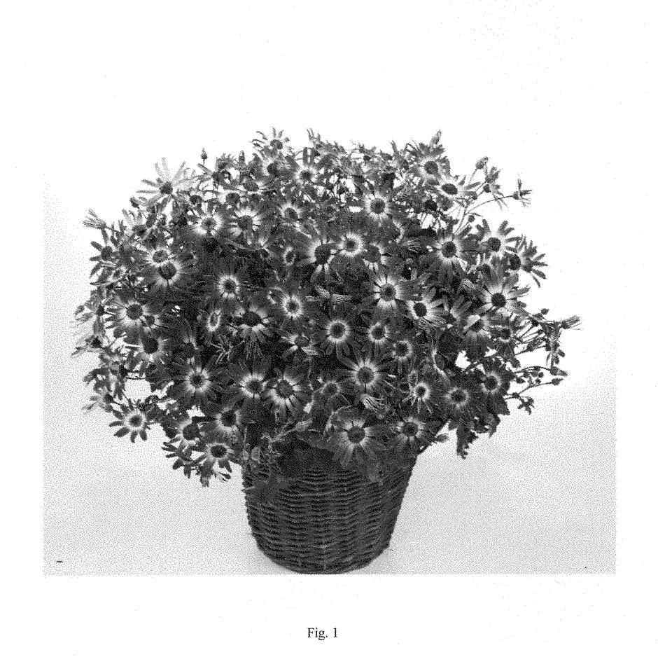

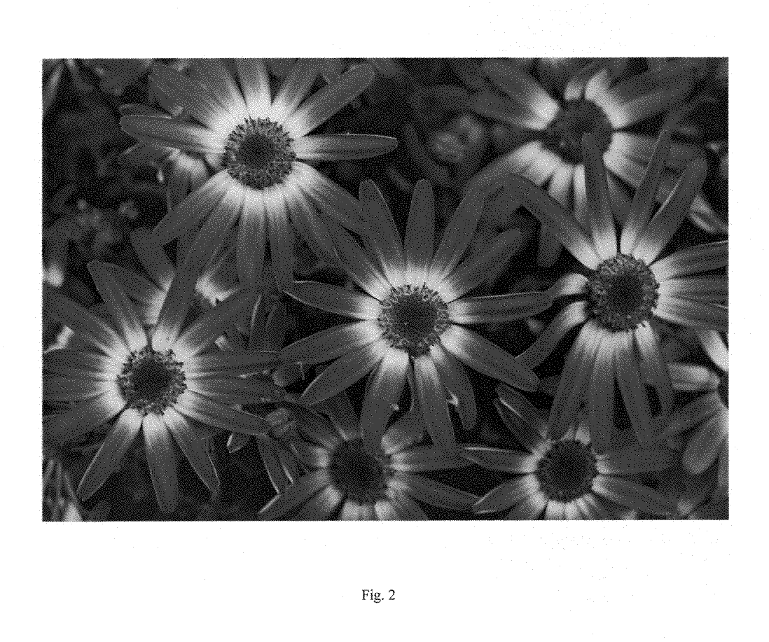

### DETAILED BOTANICAL DESCRIPTION

In the following description, color references are made to The Royal Horticultural Society Mini Colour Chart 2005 except where general terms of ordinary dictionary significance are used. The following observations and measurements describe ‘DPRIMBLUBR’ plants grown in a greenhouse in Moshav Mishmar Hashiva, Israel, under natural lighting. Measurements were taken during Spring of 2016. The plants were approximately 11 weeks old from a rooted cutting in a 13 cm pot. The growing temperature ranged from approximately 15° C. to 20° C. during the days, 7° C. to 12° C. during the nights. Measurements and numerical values represent averages of typical plant types.
Botanical classification: Pericallis hybrida ‘DPRIMBLUBR’.
Propagation:
   Time to rooting.—14 days at approximately 18° C.
   Root description.—Fine, fibrous and white, color not accurately measured with RHS chart.
Plant:
   Growth habit.—Upright.
   Height.—Approximately 24 cm.
   Plant spread.—Approximately 33 cm.
   Growth rate.—Moderate growth.
   Branching characteristics.—Freely branching.
   Length of lateral branches.—Approximately 19 cm.
   Diameter of lateral branches.—Approximately 0.3 cm.
   Texture of lateral branches.—Slightly pubescent.
   Internode length.—Approximately 2.5 cm.
   Strength of stem.—Flexible.
   Color of lateral branches.—Near Yellow-Green 146C.
   Aspect/angle of branches.—30.
   Number of leaves per lateral branch.—Approximately 6.
Foliage:
   Leaf.—Arrangement. Alternate, simple Average Length: Approximately 6 cm. Average Width: Approximately 6.5 cm. Shape of blade: Sinuate Description of Lobes: Shallowly lobed Apex: Acute Base: Sagittate Attachment: Petiolate Margin: Entire Texture of top surface: Glabrous, coarse Texture of bottom surface: Smooth, wooly, obvious venation pattern Appearance top surface: Matte Appearance bottom surface: Matte, noticeable veins, wool-like coating Leaf internode length: Approximately 4.2 cm. Color: Young foliage upper side: Near Yellow-Green 146B Young foliage under side: Near Yellow-Green 147C Mature foliage upper side: Near Yellow-Green 146A Mature foliage under side: Near Yellow-Green 147C Venation: Type: Palmate Venation color upper side: Near Yellow-Green 147B Venation color under side: Near Yellow-green 147C.
   Petiole.—Petiole: Fleshy, shiny slightly ribbed longwise, flex Length: 1.8 cm. Diameter: 0.2 cm Pubescence: No Color: Near Yellow-Green 147C.
Flower:
   Bloom period.—Late Winter to early Summer in Moshav, Mishmar Hashiva, Israel.
   Bud.—Bud shape: Globular Bud length: Approximately 0.5 cm. Bud diameter: Approximately 0.3 cm. Bud color: Near Green 138B.
   Inflorescence.—Form: Composite inflorescences positioned above the foliar plane.
   Flower.—Diameter of entire flower: Approximately 3.7 cm. Depth of flower: Approximately 0.4 cm. Width of disc: Approximately 1 cm. Depth of disc: Approximately 0.5 cm.
   Ray florets.—Number of Ray Florets: Average 13 Length: Approximately 2 cm. Width; Approximately 0.7 cm. Apex shape: Rounded Base: Attenuate Margin: Entire.
   Color.—Ray Florets: Upper surface at first opening: Near Violet N88A RHS and from White 155B at base Upper surface at maturity: Near Violet N88A RHS and from White 155C at base Upper surface at fading: Near Violet N88B RHS and White 155C at base Under surface at first opening: Near Violet-blue 91B RHS and White 155A at base Under surface at maturity: Near Violet N88C RHS and White 155C at base Under surface at fading: Near Violet N88C BRHS and White 155B at base Disc Florets: Number of Disc Florets: 120 Length: Approximately 0.7 cm. Width: Approximately 0.1 cm. Shape: Tubular Margin: Entire Color: At first opening: Near White 155B and Violet-Blue 96A At maturity: Near White 155A and Violet-Blue 96A At fading: Near White 155A and Violet-Blue 96A.
   Fragrance.—None.
   Phyllaries/involucral bracts.—Number: Approximately 14 Sepal length: Approximately 0.6 cm. Sepal width: Approximately 0.2 cm. Sepal texture: Smooth, glabrous Apex shape: Acute Margin: Entire Color: Near Yellow-Green 144A.
   Peduncle.—Peduncle length: Approximately 2.5 cm Peduncle diameter: Approximately 0.2 cm. Angle: Approximately 30 degrees from center of whorl. Color: Near Yellow-Green 148A Peduncle texture: Smooth, glabrous.
Reproductive organs:
   Disc florets.—Androecium: Present on disc florets only Stamens: About 60 Anther shape: Rounded Anther length: Approximately 0.1 cm. Anther color: Near Purple-Violet 81A Pollen quantity: Abundant.
   Gynoecium.—Pistil number: 13 Pistil shape: Forked Pistil length: Approx. 0.5 cm Style length: Approx. 0.3 cm. Style color: Near Yellow-Green. 145D Stigma shape: Decurrent Stigma color: Near Purple-Violet 80 Ovary diameter: 0.2 cm Ovary color: Near Yellow-Ureen 144A.
   Ray florets.—
   Androecium.—Absent.
   Gynoecium.—Pistil number: 1 Pistil shape: Linear Pistil length: Approx. 0.15 cm Style length: Approx. 0.15 cm. Style color: Near Yellow-Green 145D Stigma shape: Outwardly curved forked Stigma color: Near Violet-Blue N92A Ovary diameter: 0.1 cm Ovary color: Near Yellow-Green 146B.
Other characteristics:
   Disease and pest resistance.—Better resistance to powdery mildew than typical of known Pericallis varieties. Neither resistance nor susceptibility to additional normal diseases and pests of Pericallis has been observed.
   Drought tolerance and cold tolerance.—Low tolerance for cold or drought.
   Fruit/seed production.—Abundant seed production. Shape: Oblong Color: Near Brown 200B Size: Length: 0.2 cm Width: 0.05 cm.

What is claimed is:

1. A new and distinct cultivar of Pericallis plant named ‘DPRIMBLUBR’ as herein illustrated and described.
